(19) United States
(12) Patent Application Publication
(10) Pub. No.: US 2004/0146526 A1
(43) Pub. Date: Jul. 29, 2004

(54) Inhibition of NF-kappaB activation

(76) Inventors: Henry J. Windle, Dublin (IE); Dermot O'Toole, Paris (FR); Dermot Kellehor, Dublin (IE); Mohamed M. Abdel-Latif, Dublin (IE)

Correspondence Address: JACOBSON HOLMAN PLLC, 400 SEVENTH STREET N.W., SUITE 600, WASHINGTON, DC, 20004, US

(21) Appl. No.: 10/628,391

(22) Filed: Jul. 29, 2003

Related U.S. Application Data
(63) Continuation of application No. PCT/IE02/00011, filed on Jan. 30, 2002.

(30) Foreign Application Priority Data
Jan. 30, 2001 (IE) 2001/0069

(51) Int. Cl. A61K 39/02; C12N 9/02

(52) U.S. Cl. 424/190.1; 435/189

(57) ABSTRACT

A H. pylori thioredoxin protein having a seq ID No. 1 is capable of inhibiting the activation of NF-κB. The protein may be used in treating inflammation.

### FIELD OF THE INVENTION

The invention relates to a method for the prevention and reversal of NF-kappa B (NF-κB) activation in mammalian cells. In particular, the invention relates to the treatment of patients with inflammatory diseases, especially chronic inflammatory diseases such as inflammatory bowel disease, rheumatoid/autoimmune arthritis, or any disease in which the transcription factor NF-κB is transcriptionally active.

NF-κB is a eukaryotic transcription factor that exerts pleiotrophic effects on diverse genes and particularly those involved in inflammation. Transcription of the proinflammatory cytokines TNFα and IL-1β is regulated by NF-κB and both of these cytokines are documented to be increased in subjects with inflammatory bowel disease (1). Expression of the transcription factor NF-κB is also known to be increased in patients with rheumatoid/autoimmune arthritis, asthma, septic shock, lung fibrosis, glomerulonephritis, atherosclerosis, autoimmune encephalomyelitis, and other chronic inflammatory disease states. In inflammatory bowel disease, for example, increased activation of NF-κB is thought to be involved in the regulation of the inflammatory response (1, 2). Inflammatory bowel disease (IBD) is a severe chronic inflammation treated mainly by immunosuppression (3). High levels of NF-κB activation have been shown in both Crohns disease, a chronic inflammatory disease, and animal models of inflammatory bowel disease. Crohns disease is regarded as medically incurable. Treatment is aimed at inducing and maintaining remission and reducing complications. Crohns disease is usually treated with 5-aminosalicylic acid, which has topical anti-inflammatory activity in the large and small bowel (4).

Novel inhibitors of NF-κB are currently under development for the treatment of inflammatory diseases such as asthma, rheumatoid/autoimmune arthritis and inflammatory bowel disease (5). Local administration of NF-κB p65 antisense phosphorothioate oligonucleotides in inflammatory bowel disease has been shown to abrogate the clinical and histological signs of colitis (6). Other recent developments in the treatment and management of inflammatory bowel disease have been reviewed by Stein & Hanauer (3).

The majority of anti-inflammatory drugs fall into two categories, non steroidal anti inflammatory drugs (NSAIDs) and derivatives of corticosteroids. Efficacy and toxicity vary. Regular users of NSAIDs are at serious risk of developing gastrointestinal disorders (7). NSAIDs work principally by interfering with the synthesis of inflammatory mediators (prostaglandins), whereas the corticosteroids have broad range effects due to their ability to regulate gene expression. Systemic use of corticosteroids is frequently associated with debilitating side effects although some corticosteroid analogues such as budesonide have less toxic side effects (8). Other classes of drugs are becoming available, for example leukotriene blockers (Singulair-Merck), which inhibit pro-inflammatory cell signalling mediated by this class of chemokines.

There is therefore an ongoing need for pharmaceuticals for the prophylaxis and/or treatment of diseases where the transcription factor NF-κB is transcriptionally active.

### STATEMENTS OF INVENTION

According to the invention there is provided a H. pylori protein or derivative or fragment or mutant or variant thereof capable of inhibiting the activation of NF-κK.

Preferably the protein is a thioredoxin or derivative or fragment or mutant or variant thereof.

We have identified a protein with the following amino acid sequence:

| | | | |
|---|---|---|---|
| MSHYIELTEE | NFESTIKKGV | ALVDFWAPWC | GPCKMLSPVI |
| DELASEYEGK | AKICKVNTDE | QEELSAKFGI | RSIPTLLFTK |
| DGEVVHQLVG | VQTKVALKEQ | LNKLLG | |

The invention also provides a thioredoxin or derivative or fragment or mutant or variant thereof containing the redox active peptide sequence CGPC capable of inhibiting the activation of NF-κB.

The invention further provides prokaryotic or eukaryotic thioredoxins having potent immune-suppressive effects.

The invention also provides polypeptides containing the redox active peptide sequence CGPC, capable of inhibiting the activation of NF-κB.

The invention also provides a H. pylori protein having the following amino acid sequence:

| | | | |
|---|---|---|---|
| MSHYIELTEE | NFESTIKKGV | ALVDFWAPWC | GPCKMLSPVI |
| DELASEYEGK | AKICKVNTDE | QEELSAKFGI | RSIPTLLFTK |
| DGEVVHQLVG | VQTKVALKEQ | LNKLLG | |

The invention further provides use of a H. pylori thioredoxin protein or derivative or fragment or variant thereof of the invention in a method for the prevention and/or treatment of inflammation, such as for the prevention and/or treatment of inflammatory bowel disease.

The invention also provides use of a H. pylori thioredoxin protein or derivative or fragment or variant thereof of the invention in a method for the prevention and/or treatment of rheumatoid/autoimmune arthritis, or other autoimmune diseases, asthma, septic shock, lung fibrosis, glomerulonephritis, atherosclerosis, autoimmune encephalomyelitis or any chronic disease wherein NF-κB is transcriptionally activated.

The invention also provides use of a H. pylori thioredoxin protein or derivative or fragment or mutant thereof of the invention in blood transfusions and soft tissue injury.

The invention also provides a H. pylori thioredoxin protein or derivative or fragment or mutant thereof of the invention for use in the preparation of a medicament in the treatment and/or prophylaxis of any chronic disease wherein NF-κB is transcriptionally activated.

The invention further provides a protein of the invention for use in the preparation of a medicament for the treatment and/or prophylaxis of any chronic disease wherein NF-κB is transcriptionally activated.

### BRIEF DESCRIPTION OF THE DRAWINGS

The invention will more clearly understood from the following description thereof given by way of example only with reference to the accompanying drawings, in which:—

FIG. 1 is an autoradiograph showing the results of an electrophoretic mobility shift assay (EMSA) showing the effect of H. pylori thioredoxin on constitutive NF-κB activity in AGS cells (an adenocarcinoma cell line);

FIG. 2 is an autoradiograph showing the results of an EMSA showing the time course of NF-κB inhibition upon treatment of AGS cells with H. pylori thioredoxin;

FIG. 3 is an autoradiograph showing the results of an EMSA showing the effects of H. pylori thioredoxin on H. pylori-induced NF-κB activation in AGS cells;

FIG. 4 is an autoradiograph showing the results of an EMSA showing the effect of H. pylori thioredoxin on NF-κB activation by various stimuli;

FIG. 5 is an autoradiograph showing the results of an EMSA showing the inhibition of H. pylori-induced NF-κB by H. pylori thioredoxin post stimulation with H. pylori; and

FIG. 6 is a SDS-PAGE gel showing the identification of AGS cell proteins reduced specifically by H. pylori thioredoxin.

FIG. 7 is a FACscan analysis demonstrating the down-regulatory effect of thioredoxin on the surface expression of CD44 and ICAM-1 on AGS cells treated with or without H. pylori.

DETAILED DESCRIPTION

We have found a method for the prevention and reversal of NF-κB activation in mammalian cells by addition of effective inhibiting amounts of a H. pylori thioredoxin either alone or in combination with a thioredoxin reductase regenerating system. The H. pylori thioredoxin may be in the form of the whole recombinant protein or a fragment or derivative or mutant or variant thereof.

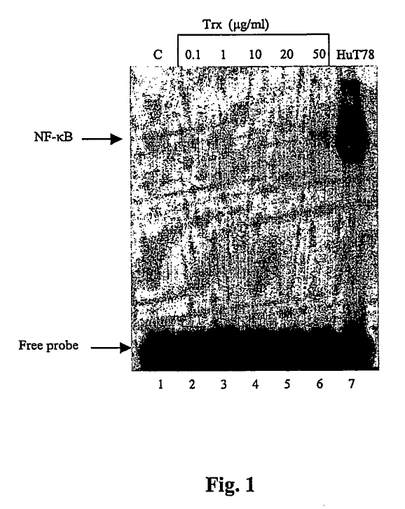

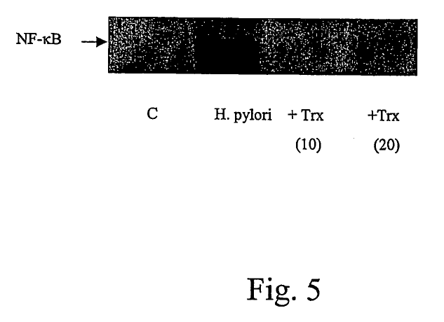

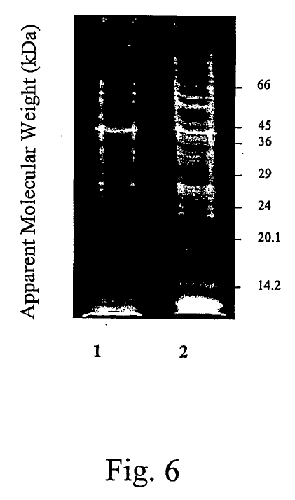

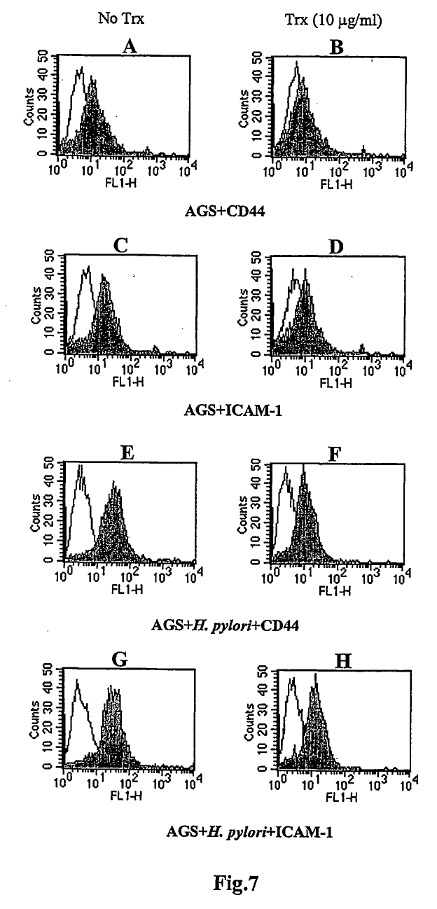

We have found that recombinant thioredoxin from the gastric pathogen H. pylori is a potent inhibitor of NF-κB activation in vitro. When added exogenously to AGS cells (an adenocarcinoma cell line) in vitro, low doses of H. pylori thioredoxin (1-20 μg/ml; 70 nM to 1.4 μM) inhibit constitutive NF-κB activity. In addition, H. pylori thioredoxin completely abrogates the pronounced NF-κB activity observed in AGS cells when NF-κB DNA binding activity is activated by a variety of external stimuli including proinflammatory cytokines and phorbol esters. H. pylori Trx was found to prevent NF-κB activation both prior to stimulation (FIGS. 1 to 4) with inducers of NF-κB and secondary to induction of NF-κB (FIG. 5). Preliminary experiments (FIG. 6) indicate that H. pylori Trx interacts specifically with target proteins in AGS cells as demonstrated by the incorporation of the thiol-specific fluorescent probe, monobromobimane, into Trx-treated AGS cells. The precise mechanism of Trx-modulated NF-κB activity has yet to be fully elucidated. H. pylori thioredoxin also down-regulates the resting and inducible surface expression of CD44 and the adhesion molecule ICAM-1 (FIG. 7)

The ability of H. pylori thioredoxin to inhibit NF-κB activation in vitro suggest a potential therapeutic utility for thioredoxin as a novel approach for the treatment of patients with chronic inflammatory disease states such as autoimmunic arthritis, asthma, septic shock, lung fibrosis, glomerulonephritis, atherosclerosis, autoimmune encephalomyelitis, cystic fibrosis, rheumatoid arthritis, systemic inflammatory response syndrome and other NF-κB-mediated inflammatory disease states.

The present invention provides a protein, H. pylori thioredoxin, comprising a redox-active motif (CGPC), (cysteine-glycine-proline-cyseine), capable of inhibiting activation of the transcription factor NF-κB.

The protein has the amino acid sequence:

!SEQ ID NO 1

| | | | |
|---|---|---|---|
| MSHYIELTEE | NFESTIKKGV | ALVDFWAPWC | GPCKMLSPVI |
| DELASEYEGK | AKICKVNTDE | QEELSAKFGI | RSIPTLLFTK |
| DGEVVHQLVG | VQTKVALKEQ | LNKLLG | |

In the above sequence, individual amino acids are represented by the single letter code commonly used in the field.

The present invention also includes within its scope peptides derived from H. pylori thioredoxin identified above where such derivatives have redox-activity or where such derivatives inhibit NF-κB activation. These derivatives will normally be peptide fragments of the native protein which include the redox-active motif, but can also be functionally equivalent variants of the recombinant thioredoxin modified by well known techniques such as site-directed mutagenesis. For example, it is possible by such techniques to substitute amino acids in a sequence with equivalent amino acids. Groups of amino acids known to be normally equivalent are:

| | | | | | |
|---|---|---|---|---|---|
| (a) | A | S | T | P | G; |
| (b) | N | D | E | Q; | |
| (c) | H | R | K; | | |
| (d) | M | L | I | V; and | |
| (e) | F | Y | W. | | |

Thioredoxin variants can be obtained by conventional gene engineering technology. For example, the amino acid sequence and base sequence of thioredoxin are known and described in numerous documents in the scientific literature. Based on the prior art documents, cDNA encoding natural thioredoxin can be obtained from an appropriate cDNA library. A variant can then be obtained by, for example, site-directed mutagenesis (9).

The recombinant protein is preferably used rather than the native protein as the native protein is generally not present in sufficient abundance.

The term derivative, fragment and mutant are understood to have the same meaning as commonly understood by one skilled in the art to which the invention belongs. A derivative is a chemical modification. A fragment may range in size from 4 amino acids to the entire amino acid sequence minus one residue. A mutant may have one or more changes in the molecular sequence of the gene. Derivatives, fragments or mutants have modifications on the protein however they retain the essential biological characteristics and activities of the protein.

The thioredoxin of the invention may be produced by isolation from H. pylori, using conventional purification techniques. However, it is recognised that for production of the protein in commercial quantities, production by synthetic routes is desirable. Such routes include the stepwise solid phase approach and production using recombinant DNA techniques. The latter route is preferred.

Stated generally, the production of thioredoxin by recombinant DNA techniques involves the transformation of a suitable host organism or cell with an expression vector including a DNA sequence coding for thioredoxin, followed by the culturing of the transformed host and subsequent recovery of the expressed thioredoxin. Such techniques are described generally in Sambrook et al. Molecular Cloning, 2nd edition, Cold Spring Harbour Press (1987).

The redox protein thioredoxin and the associated enzyme thioredoxin reductase (TR) constitute a thiol-dependent reduction-oxidation system that can catalyse the reduction of certain proteins by NADPH (10).

In its primary aspect, the present invention is directed to the provision of thioredoxin which is protective against inflammation. Subjects which are susceptible to inflammation are mammals including humans.

The concentration of thioredoxin which can be used ranges from about 1 μM to about 30 μM. The optimal concentration for intact reduced H. pylori thioredoxin appears to be a least 10 μM.

The thioredoxin compound may be orally administered to a patient requiring such treatment on a regular basis over an extended period of time. Alternatively, the compound may be administered directly to the localised site of inflammation.

It should be recognised that the precise level of thioredoxin can be readily ascertained by a person skilled in the art in light of the present invention.

Thioredoxin and thioredoxin derived derivatives, fragments or mutants thereof may be administered directly, in the form of a formulation or any other pharmaceutically acceptable manner. Preferably such formulation includes an ingestable carrier which is a pharmaceutically acceptable carrier such as a capsule, tablet or powder. The formulation may also include a drug entity.

While the invention is broadly as defined above, it will be appreciated by those persons skilled in the art that it is not limited thereto but that it also includes embodiments of which the following description provides examples.

Materials and Methods Used in the Purification of Thioredoxin from H. pylori and Inhibition of NF-κB Activity.

Materials 2′,5′-ADP-agarose, Cibacron Blue 3GA, iminodiacetic acid-Sepharose 6B, ρ-aminobenzamidine-agarose, DTT (1,4-dithio-DL-threitol), E. coli thioredoxin and anti-E. coli thioredoxin were obtained from Sigma Chemical Co. Ltd., Poole, Dorset, U.K. Sephacryl S-300 was obtained from Pharmacia Biotech, Uppsala, Sweden. Isopropyl-β-D-thiogalactoside, NADPH, NADP+, and NADH were obtained from Boehringer Mannheim, Bell Lane, Lewes, East Sussex, UK. DEAE-52 was purchased from Whatman (Maidstone, UK). Factor Xa was purchased from New England Biolabs, Hertfordshire, U.K. All buffer reagents for SDS-PAGE were prepared in deionised water. The human gastric cancer cell line AGS and HuT 78, sezary lymphoma cells, were obtained from the European collection of Animal Cell Cultures (ECACC, Porton Down, Salisbury, UK). RPMI 1640 medium, fetal calf serum, penicillin, streptomycin, L-glutamine, Hank's Balanced salt solution (HBSS) and trypsin were obtained from GIBCO BRL, life technologies Renfrewshire, Pasiley, Scotland. NF-κB consensus oligonucleotide was from Promega, poly(dI-dC) was from Pharmacia, Biosystems, Milton Keynes, UK. [γ³²P]ATP (35 pmol, 3000 Ci/mmol) was from Amersham International (Aylesbury, UK). Bovine albumin, ammonium persulphate, Nonidet P-40, PMA, IL-1 and PMSF were obtained from Sigma (Poole, Dorset, UK and St. Louis, Mo., USA). All other chemicals were of analytical reagent grade.

Western blotting and SDS-PAGE. Discontinuous SDS-PAGE was performed essentially as described previously in Sambrook et al. Proteins from SDS-PAGE gels were electroblotted (0.9 mA/cm² for 1 h) to polyvinylidene difluoride membrane (Gelman) using a semi-dry blotting apparatus (LKB/Pharmacia), essentially as described by Towbin et al. (11). Immunoblots were processed and developed by enhanced chemiluminesence as described previously (10). For N-terminal sequencing the protein was electroblotted to ProBlott and stained briefly with freshly prepared amido black.

Protein measurements. Protein was measured using standard procedures with bovine serum albumin as the protein standard.

Bacterial strain and growth conditions. The reference strains of H. pylori used in this study (NCTC 11638 and 11637) were obtained from the National Collection of Type Cultures, Public Health Laboratory, London, U.K. All components for H. pylori culture media were obtained from Oxoid, Unipath Ltd., Basingstoke, Hampshire, U.K. H. pylori was grown under microaerobic conditions (Oxoid Campylobacter system, 5% O₂, 10% CO₂) for 4 days on 7% lysed horse blood Columbia agar at 37° C. Bacteria were harvested into RPMI medium without antibiotics and resuspended to yield a concentration of 6×10⁸ organisms/ml and used immediately.

Purification of thioredoxin reductase (TR). Agar-grown H. pylori was suspended in buffer A (20 mM Tris-HCl, pH 7.5) and subjected to sonication (4×1 min bursts) on ice using a Branson sonifier 450. After centrifugation to remove intact cells and cellular debris (12, 000×g, 10 min, 4° C.) the resulting supernatant was applied to a DEAE cellulose column (3.5×16 cm) equilibrated in buffer A. Thioredoxin reductase activity was eluted with a gradient (300 ml) of KCl (0-0.35 M) in buffer A. Active fractions were pooled, dialyzed against buffer B (50 mM Tris-HCl, pH 7.5) and applied to a Cibacron Blue 3GA column (1×3 cm). TR was eluted with a gradient of KCl (0-0.4 M). Active fractions were pooled, dialyzed against buffer B and applied to a small 2′,5′-ADP agarose column (1 ml). Thioredoxin reductase was eluted upon application of 0.2 M KCl. The ion exchange and dye affinity chromatography steps were performed at room temperature and the ADP-Sepharose step was done at 4° C.

Gel filtration chromatography. A sonicate of H. pylori was prepared as described above and 0.5 ml (˜10 mg protein/ml) of the material was applied to a column (diameter 1.5 cm; height 29.7 cm) of Sephacryl S-300 superfine (Pharmacia) equilibrated with phosphate buffered saline (pH 7.5) containing NaN₃ (0.02%, w/v). The protein was eluted with this same buffer (8.5 cm/h) and the collected fractions were assayed for both TR activity and total protein. The column was first calibrated with proteins of known molecular size (Pharmacia). Gel filtration over Sephadex G-50 (Pharmacia) was performed also in phosphate buffered saline (PBS).

Measurement of thioredoxin reductase activity. Thioredoxin reductase activity was assayed at 25° C. in 0.1 M potassium phosphate buffer (pH 7.5) containing EDTA (1 mM), DTNB (5 mM) and NADPH (0.2 mM) in a final volume of 1.0 ml. The reaction was initiated by the addition of enzyme and the progress of the reaction was monitored by the increase in absorbance at 412 nm in a Pye Unicam 5625 spectrophotometer. One unit of enzyme activity is defined as the amount of enzyme required to oxidize one μmol of NADPH per minute at 25° C., pH 7.5. Activity was calculated as μmol NADPH oxidized/min in accordance with the relationship ΔA412/(13.6×2). Thioredoxin reductase activity was assayed also using a minor modification of the insulin reduction assay (12). The reaction mixture consisted of 0.1 M potassium phosphate buffer (pH 7.0) containing EDTA (1 mM), insulin (0.1 mg/ml), NADPH (0.2 mM) and H. pylori histidine-tagged Trx (2 μM) in a final volume of 1 ml. The reaction was initiated by the addition of the enzyme to the mixture at 25° C. and the oxidation of NADPH was monitored at 340 nm. The amount of NADPH oxidized was determined from the relationship ΔA340/6.2.

Purification of native H. pylori Trx. Thioredoxin (Trx) was purified by a combination of ion exchange chromatography on DEAE cellulose and gel filtration over Sephadex G-50. Fractions containing Trx were identified using the spectrophotometric insulin reduction assay (12).

Expression and purification of recombinant H. pylori Trx. Transformants of E. coli BL21(DE3)pLysS with plasmid pET-16b (Novagen) containing the Trx gene (HP 824) were grown at 37° C. in LB broth supplemented with ampicillin (100 μg/ml) and chloramphenicol (30 μg/ml). H. pylori Trx was expressed as an N-terminal decahistidine fusion protein in E. coli. The gene coding for Trx was amplified by PCR using Expand™ (Boehringer Mannheim), using the amplification conditions recommended by the manufacturer. Under these conditions a single product was obtained and this was cloned into the expression plasmid via the BamHI and NdeI restriction sites. The following primers were used: forward primer, 5′-CGCCATATGAGTCACTATATTGAATTAAC-3′; reverse primer 5′-CGCGGATCCGCCTAAGAGTTTGTTCAATTG-3′. Overexpression of the fusion protein was induced by adding 1 mM isopropyl-β-D-thiogalactoside at exponential phase and the incubation continued for 3 h at 37° C. The induced cells were harvested by centrifugation (10,000×g, 15 min, 4° C.), washed once with 50 mM Tris HCl (pH 7.5) and subjected to sonication (3×1 min). The soluble fusion protein was purified to homogeneity by metal chelate chromatography on a Ni²⁺ column (3 ml) according to the manufacturer's instructions. The protein was eluted with 0.4 M imidazole in 20 mM Tris HCl (pH 7.5) containing 0.5 M NaCl. Typically, 2-3 mg of homogenous Trx/100 ml culture was obtained by this procedure. Both the histidine tagged fusion protein and the recombinant Trx obtained after cleavage of the histidine tail by Factor Xa were indistinguishable in their spectroscopic properties and redox behaviour.

Sequence analysis Multiple sequence alignments were made with the Clustal program. Amino-terminal sequence analysis of purified H. pylori Trx and TR was performed by Ms. Aine Healy at the National Food Biotechnology Centre, University College Cork using an Applied Biosystems automated sequencer.

Cell culture conditions AGS cells were grown in RPMI 1640 culture medium supplemented with 10% fetal calf serum, 100 units/ml penicillin, 1001 μg/ml streptomycin and 2 mM L-glutamine at 37° C. (5% CO₂). For experiments, AGS cells were seeded at a density of 1×10⁵ cells/ml culture medium in 6-well plates and left overnight until confluent prior to experiments.

Coculture of AGS cells with H. pylori and other stimuli Confluent AGS cells were cocultured with or without H. pylori (6×10⁸ cfu/ml) or exposed to the cytokines interleukin-1beta (IL-1 β) (10 ng/ml) and tumor necrosis factor-alpha (TNF-α) (20 ng/ml) or the mitogen phorbol 13-myristate 12-acetate (PMA) (20 ng/ml).

Preparation of nuclear extracts Nuclear extracts were prepared from unstimulated and stimulated AGS cells as described. Briefly, the cells were washed twice in ice-cold PBS, harvested by scraping with a cell scraper, and transferred into centrifuge tubes on ice. The cells were pelleted by centrifugation at 1400 rpm for 5 min and washed once in (1 ml) buffer A (10 mM HEPES (pH 7.9), 1.5 mM MgCl₂, 10 mM KCl, 0.5 mM phenylmethylsulfonylfluride (PMSF) and 0.5 mM dithiothreitol (DTT) and centrifuged at 10,000 rpm for 10 min. The cell pellet was then resuspended in (20 μl) buffer A containing 0.1% Nonidet P-40 (buffer B) for 10 min on ice and lysed cells were centrifuged at 10,000 rpm for 10 min. The supernatant was discarded and the nuclear pellet was extracted with (15 μl) buffer C (20 mM HEPES (pH 7.9), 420 mM NaCl, 1.5 mM MgCl₂, 0.2 mM EDTA, 25% glycerol and 0.5 mM PMSF) for 15 min on ice. After incubation, the nuclei were centrifuged at 10000 rpm for 10 min and the supernatant was diluted with 4 volumes of buffer D (10 mM HEPES (pH 7.9), 50 mM KCl, 0.2 mM EDTA, 25% glycerol and 0.5 mM PMSF). The nuclear extracts were used immediately or stored at −70° C. until required. The protein concentration was determined on nuclear extracts by the method of Bradford.

Electrophoretic mobility-shift assays (EMSA) For binding assays, nuclear extracts (4 μg of protein) were incubated with 0.10000 cpm of the ³²P-labelled oligonucleotide (22 bp) comprising the consensus sequence of the NF-κB (5′-AGT TGA GGG GAC TTT CCC AGG C-3′)(3′-TCA ACT CCC CTG AAA GGG TCC G-5′) that had been previously labelled with (Y-³²P)ATP at the 5′-ends with T4 polynucleotide kinase in 20 μl binding reaction in binding buffer (10 mM Tris, pH 7.5, 40% glycerol, 5 mM DTT, 1 mM EDTA, 100 mM NaCl and 0.1 mg/ml nuclease free bovine serum albumin) in the presence of 2 μg of poly(dI-dC) as non specific competitor. The reaction mixture was then incubated for 30 min at room temperature after the addition of the probe DNA. The binding reaction was terminated using a loading dye prior to adding the samples to the gels. The DNA-protein complexes were separated on 5% polyacrylamide gels (pre run at 80 V for 30 min) at 150 V for 1-2 h at room temperature. After electrophoresis was performed, the gels were dried and autoradiographed at −70° C. for 24-36 h with intensifying screens.

Cell proliferation and toxicity assays AGS cells (1×10⁵ cells/ml) were cultured in 96-well plates in triplicate overnight at 37° C. The cells were then incubated with or without thioredoxin for various periods of time, as indicated where appropriate, at 37° C. To the cultured cells, 20 μl of freshly prepared PMS/MTS solution was added to each well and the plates were incubated for 4 h at 37° C. The absorbance of these wells was read at 490 nm using an ELISA plate reader. The average of the triplicate readings was taken for each sample. Under the experimental conditions and in the range of thioredoxin concentrations used, the cell viability was greater than 90%.

Flow cytometry analysis AGS cells were grown to confluence on 6-well plates and then incubated with or without thioredoxin (10 μg/ml) for 2 h at 37° C. The cells were then stimulated with H. pylori for 24 h at 37° C. The cells were washed with PBS and incubated for 30 min with antibodies to CD44 (L3D.1) and ICAM-1 at room temperature followed by washing and labelling with fluorescein isothiocynate (FITC)-conjugated rabbit F(ab)₂′ anti-mouse IgG (Dakopotts, Glostrup, Denmark). Samples were analysed by flow cytometry in a FAC scan (Becton Dickinson, Mountain View, Calif.).

### EXAMPLE 1

The effect of thioredoxin on constitutive NF-κB in AGS cells was examined. AGS cells were treated as described above with different concentrations of Trx (0.1 g/ml, 1.0 μg/ml, 10 μg/ml, 20 μg/ml and 50 μg/ml) for 2 hours. A positive control comprising HuT78 cells was used. Hut78 cells have high constitutive levels of NF-κB. The samples were analysed by EMSA and the results are shown in FIG. 1. Lane 1(C) represents a control of untreated resting AGS cells. The level of NF-κB activity decreases as the concentration of Trx increase to 20 μg/ml. At 50 μg Trx/ml NF-κB activity appears to increase. Using elevated extracellular amounts of thioredoxin was inimical to cell viability as judged by phase contrast microscopy and by staining cells with ethidium bromide/acradine orange. The increase in NF-κB DNA-binding activity in this instance is likely due to stresses imposed on the cells as a consequence of exposure to elevated amounts (>20 μg/ml) of thioredoxin.

### EXAMPLE 2

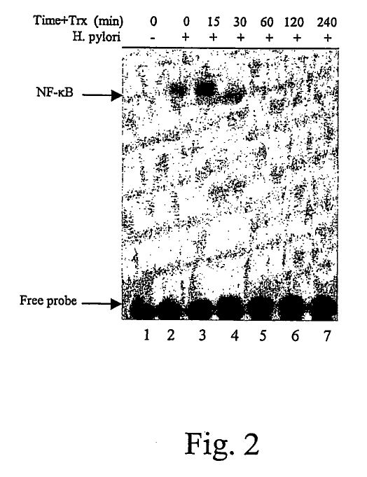

The inhibition of NF-κB by Trx over time in AGS cells was examined by pre-incubating AGS cells as described above with Trx (10 μg/ml) for different time period (0 mins, 15 mins, 30 mins, 60 mins, 120 mins and 240 mins) After treatment with Trx the cells were stimulated for 2 hrs with H. pylori (6×10⁸ cfU/ml). Nuclear extracts were prepared and analysed for NF-κB DNA-binding activity by EMSA. The results are shown in FIG. 2 where it can be seen that AGS cells must be exposed to Trx for at least 30 min prior to stimulation with H. pylori to block H. pylori-induced NF-κB DNA-binding activity. Control untreated AGS cells are shown in lane 1 and H. pylori treated AGS cells are shown in lanes 2-7.

### EXAMPLE 3

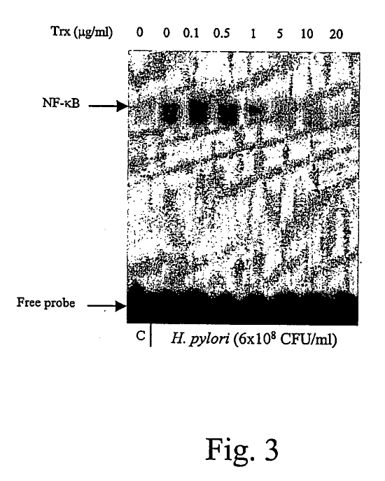

The dose-dependent effect of Trx on H. pylori-induced NF-κB activation is shown in FIG. 3. AGS cells were treated for 2 hrs with increasing amounts of Trx (0.1 μg/ml, 0.5 μg/ml, 1.0 μg/ml, 5.0 μg/ml, 10 μg/ml and 20 μg/ml). After treatment the cells were stimulated with H. pylori (6×10⁸ cfu/ml) for a further 2 hrs and nuclear extracts were prepared and NF-κB DNA-binding activity was analysed by EMSA. FIG. 3 shows that the DNA-binding activity of NF-κB decreased when the cells were pretreated with increasing amounts of Trx. Lane C shows the resting levels of NF-κB in untreated resting AGS cells.

### EXAMPLE 4

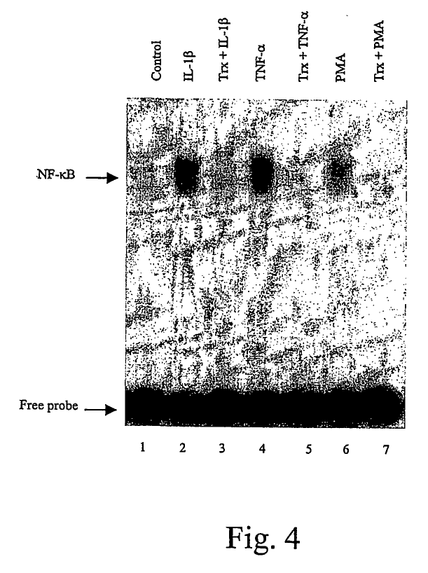

The effect of thioredoxin on NF-κB DNA-binding activity in response to stimulation by cytokines and mitogens is shown in FIG. 4. AGS cells were pre-treated with Trx (10 ug/ml) for 2 hrs and then stimulated with TNFα (20 ng/ml), IL-1β (10 ng/ml) or PMA (20 ng/ml) for an additional 2 hrs. Nuclear extracts were prepared and NF-κB-DNA-binding activity was analysed by EMSA.

As can be seen in FIG. 4 the cells treated with the pro-inflammatory cytokines TNFα, IL-1 β and the mitogen PMA showed pronounced NF-κB DNA-binding activity in the absence of Trx. However, this activity was almost completely inhibited when the AGS cells were pre-treated with Trx. Control untreated AGS cells are shown in lane 1.

### EXAMPLE 5

The inhibition of H. pylori-induced NF-κB by Trx was examined with the results shown in FIG. 5. AGS cells were co-cultured with H. pylori (6×10⁸ cfu/ml) to induce NF-κB activation (lane H. pylori). Subsequent to the activation of NF-κB, exogenous Trx (10 μg/ml or 20 μg/ml) was added to the stimulated cells (lanes+Trx (10) and +Trx (20), respectively). Nuclear extracts were prepared as described above and analysed for NF-κB DNA-binding activity by EMSA. Control untreated AGS cells are shown in lane C.

FIG. 5 shows that the NF-κB DNA-binding activity in cells stimulated by H. pylori could be reversed by the subsequent addition of Trx.

### EXAMPLE 6

FIG. 6 shows the identification of AGS cell proteins reduced specifically by Trx. A sonicated preparation of AGS cells was incubated either alone (lane 1) or in the presence of Trx (10 μg/ml) (lane 2) for 1 hr at 37° C. prior to incubation with the thiol-specific probe, monobromobimane (mBBr) for an additional 20 mins. The mixture was dialysed briefly (30 min) to remove excess mBBr and salt prior to acetone precipitation and subsequent separation of the labelled proteins on a 12.5% SDS-PAGE gel. Protein incorporation of mBBr were visualised by exposing the gel to UV light at 362 nm.

The results indicate that H. pylori Trx interacts specifically with target proteins in AGS cells.

### EXAMPLE 7

FIG. 7. Shows the effect of Trx on CD44 and ICAM-1 expression in AGS cells. Panels A-D show the results of FACScan analyses of AGS cells (5×10⁵ cells/ml) treated with or without Trx (10 μg/ml) for 24 hrs prior to staining with FITC-conjugated anti-CD44 mAb (L3D.1) (panels A, B) or anti-ICAM-1 (panels C, D) or FITC-labelled isotype matched control (anti-IE; unshaded peaks). Panels E-H show the effect of Trx on H. pylori-induced CD44 and ICAM-1 expression on AGS cells. AGS cells were pre-treated with Trx (10 μg/ml) for 24 hrs prior to co-incubation with H. pylori (8×10⁶ cfu/ml) for a further 24 hrs. Cells were stained with the same antibodies described above.

The results show that transactivation of the NFkB responsive genes encoding CD44 and ICAM-1 is down-regulated by H. pylori Trx as is the inducible expression of these adhesion molecules.

The protein or polypeptide of the invention can be used for prophylaxis or treatment of various disease states in animals or, especially, humans. For example, the composition may be used in the treatment of inflammatory diseases, such as chronic inflammatory diseases, for example inflammatory bowel disease, rheumatoid/autoimmune arthritis or any disease in which the transcription factor NF-KB is transcriptionally active. The mode of administration will depend on the nature of the disease and the site to which the material is to be applied or delivered. In the case of inflammation, the material would be administered or delivered as close as possible to the site of inflammation. For example, the administration may be by way of a localised injection. The administration may, for example, be by an oral, rectal, vaginal, nasal, or sublingual topical route or systemically.

The pharmaceutical composition of the invention may be administered alone or in combination with any suitable carrier, adjuvant, agent and/or drug. The formulation of such compositions will be well known in the art.

The composition may be administered at any desired dosage rate, typically in the range of 1 μg/kg to 100 mg/kg.

The invention is not limited to the embodiments therein before described which may be varied in detail.

### REFERENCES

1. Schreiber S, Nikolaus S, Hampe J. Activation of nuclear factor kappa B inflammatory bowel disease. Gut 1998 42(4):477-84

2. Neurath M F. Pathogenesis of inflammatory bowel disease: transcription factors in the spotlight. Gut 1998 42(4):458-9

3. Stein R B, Hanauer S B. Medical therapy for inflammatory bowel disease. Gastroenterol. Clin North Am 1999 28(2):297-321

4. Messori A, Brignola C, Trallori G, Rampazzo R, Bardazzi G. Belloli C, d'Albasio G, De Simone G, Martini N. Am J Gastroenterol. 1994; 89: 692-698.

5. Sands B E. Novel therapies for inflammatory bowel disease. Gastroenterol Clin North Am 1999 28(2):323-51

6. Neurath M F., Petterson S, Meyer zum Buschenfelde K H, Strober W, (1996) Nat. Med. 2 998-1004.

7. Faucheron J L. Toxicity of non-steroidal anti-inflammatory drugs in the large bowel. Eur J Gastroenterol Hepatol 1999 11(4):389-92

8. Sachar D B. N Engl J. Med. 1994; 331: 873-874.

9. Zoller, M J. & Smith M. (1982) Nucleic Acid Research 10, 6487-6500

10. Windle, H. J. and Kelleher, D. (1997) Infect. Immun. 65, 3132-3137

11. Towbin, H., Staehelin, T. and Gordon, J. (1979) Proc. Natl. Acad. Sci. USA 76, 4350-4354

12. Luthman, M. and Holmgren, A. (1982) Biochemistry 21, 6628-6633

1. A H. pylori protein or derivative or fragment or mutant or variant thereof capable of inhibiting the activation of NF-κB.

2. A H. pylori protein as claimed in claim 1 wherein the protein is a thioredoxin or derivative or fragment or mutant or variant thereof.

3. A H. pylori protein as claimed in claim 1 wherein the protein has the following amino acid sequence:

| | | | |
|---|---|---|---|
| MSHYIELTEE | NFESTIKKGV | ALVDFWAPWC | GPCKMLSPVI |
| DELASEYEGK | AKICKVNTDE | QEELSAKFGI | RSIPTLLFTK |
| DGEVVHQLVG | VQTKVALKEQ | LNKLLG | |

4. A thioredoxin or derivative or fragment or mutant or variant thereof containing the redox active peptide sequence CGPC.

5. Prokaryotic or eukaryotic thioredoxins having potent immune-suppressive effects.

6. Polypeptides containing the redox active peptide sequence CGPC.

7. A H. pylori protein having the following amino acid sequence:

| | | | |
|---|---|---|---|
| MSHYIELTEE | NFESTIKKGV | ALVDFWAPWC | GPCKMLSPVI |
| DELASEYEGK | AKICKVNTDE | QEELSAKFGI | RSIPTLLFTK |
| DGEVVHQLVG | VQTKVALKEQ | LNKLLG | |

8. A derivative or fragment or mutant or variant of the protein of claim 7.

9. Use of a H. pylori thioredoxin protein or derivative or fragment or variant thereof as claimed in claim 1 in the prevention and/or treatment of inflammation.

10. Use of a H. pylori thioredoxin protein or derivative or fragment or variant thereof as claimed in claim 9 in the prevention and/or treatment of inflammatory bowel disease.

11. Use of a H. pylori thioredoxin protein or derivative or fragment or variant thereof as claimed in claim 9 in the prevention and/or treatment of rheumatoid/autoimmune arthritis.

12. Use of a H. pylori thioredoxin protein or derivative or fragment or variant thereof as claimed in claim 9 in the prevention and/or treatment of any chronic disease wherein NF-κB is transcriptionally activated.

13. Use as claimed in claim 11 for the prevention and/or treatment of any one or more of autoimmune arthritis or other autoimmune diseases, asthma, septic shock, lung fibrosis, glomerulonephritis, atherosclerosis or autoimmune encephalomyelitis.

14. Use of a H. pylori thioredoxin protein or derivative or fragment or mutant or variant thereof as claimed in any preceding claim in soft tissue injury.

15. A H. pylori thioredoxin protein or derivative or fragment or mutant or variant thereof as claimed in claim 1 for use in the preparation of a medicament for the treatment and/or prophylaxis of any chronic disease wherein NF-κB is transcriptionally activated.

16. A protein as claimed in claim 1 for use in the preparation of a medicament for the treatment and/or prophylaxis of any chronic disease wherein NF-K B is transcriptionally activated.
